(19) United States
(12) Patent Application Publication
(10) Pub. No.: US 2022/0401697 A1
(43) Pub. Date: Dec. 22, 2022

(54) MOVABLE SHAFT AND MEDICAL DEVICE COMPRISING MOVABLE SHAFT

(71) Applicant: TATSUTA ELECTRIC WIRE & CABLE CO., LTD., Higashiosaka-shi, Osaka (JP)

(72) Inventor: Junichi INOUE, Higashiosaka-shi, Osaka (JP)

(21) Appl. No.: 17/640,192

(22) PCT Filed: Sep. 3, 2020
(86) PCT No.: PCT/JP2020/033352

§ 371 Date: Mar. 3, 2022

(30) Foreign Application Priority Data
Sep. 4, 2019 (JP) 2019-161293

(51) Int. Cl. A61M 25/01

(52) U.S. Cl. CPC A61M 25/0138; A61M 25/0147

(57) ABSTRACT

A movable shaft includes a tubular member. The tubular member has a proximal end and a distal end in a longitudinal direction of the tubular member. The tubular member has a bent portion extending along the longitudinal direction and allowed to be bent. Flexibility of the bent portion increases from a side of the proximal end to a side of the distal end.

### TECHNICAL FIELD

The present invention relates to a movable shaft and a medical instrument having the movable shaft.

### BACKGROUND ART

PTL 1 (National Patent Publication No. 2006-507073) describes a tube used for a medical instrument (hereinafter, it is simply referred to as a “tube”). Notches are formed on an outer peripheral surface of the tube described in PTL 1. The notches are arranged at intervals along the longitudinal direction of the tube. Since flexibility is imparted to a portion where the notches are formed, the tube described in PTL 1 can be bent at this portion.

### CITATION LIST

### Patent Literature

PTL 1: National Patent Publication No. 2006-507073

### SUMMARY OF INVENTION

### Technical Problem

In the tube described in PTL 1, the width of the notches in the longitudinal direction of the tube increases from a side of the distal end to a side of the proximal end. That is, in the tube described in PTL 1, the flexibility of the portion where the notches are formed increases from the side of the distal end to the side of the proximal end. According to findings by the present inventors, the tube described in PTL 1 has room for improvement in controllability of a curved shape in a bent portion.

The present invention has been made in view of the above-described problem of the prior art. More specifically, the present invention provides a movable shaft having improved controllability of a curved shape in a bent portion of a tubular member, and a medical instrument having the movable shaft.

### Solution to Problem

A movable shaft according to one aspect of the present invention includes a tubular member. The tubular member has a proximal end and a distal end in a longitudinal direction of the tubular member. The tubular member has a bent portion extending along the longitudinal direction and allowed to be bent. Flexibility of the bent portion increases from a side of the proximal end to a side of the distal end.

In the movable shaft, the tubular member may have an inner peripheral surface and an outer peripheral surface. The bent portion includes a plurality of through holes disposed in a row at intervals along the longitudinal direction, the through holes penetrating the tubular member from the outer peripheral surface toward the inner peripheral surface. Pitches between two through holes adjacent to each other in the longitudinal direction may decrease from the side of the proximal end to the side of the distal end.

In the movable shaft, at least one of a width of the through hole in the longitudinal direction and a width of the through hole in a circumferential direction of the tubular member may increase from the side of the proximal end to the side of the distal end.

The movable shaft may further include a pull wire disposed along the longitudinal direction so as to face the through hole.

A medical instrument according to one aspect of the present invention includes the movable shaft. The medical instrument according to the one aspect of the present invention is configured such that the bent portion is bent by pulling the pull wire.

### Advantageous Effects of Invention

According to the movable shaft and the medical instrument having the movable shaft according to one aspect of the present invention, it is possible to improve controllability of the curved shape in the bent portion of the tubular member.

### BRIEF DESCRIPTION OF DRAWINGS

FIG. 1 is a plan view of a movable sheath 100.

FIG. 2 is a plan view of a movable shaft 10.

FIG. 3 is a cross-sectional view taken along line III-III in FIG. 2.

FIG. 4 is an enlarged plan view of a bent portion 11e of a first tubular member 11.

FIG. 5 is a side view of first tubular member 11 as viewed from a direction V in FIG. 4.

FIG. 6 is a side view of first tubular member 11 as viewed from a direction VI in FIG. 4.

FIG. 7 is a cross-sectional view taken along line VII-VII in FIG. 4.

FIG. 8 is a side view of first tubular member 11 according to a first modification.

FIG. 9A is an enlarged plan view of first tubular member 11 according to a second modification.

FIG. 9B is a cross-sectional view taken along line IXB-IXB in FIG. 9A.

FIG. 10 is an enlarged plan view of first tubular member 11 according to a third modification.

FIG. 11A is a cross-sectional view taken along line XIA-XIA in FIG. 10.

FIG. 11B is a cross-sectional view taken along line XIB-XIB in FIG. 10.

FIG. 12 is a side view of first tubular member 11 at bent portion 11e in a movable sheath 200.

FIG. 13 is a side view of first tubular member 11 at bent portion 11e in a movable sheath 300.

FIG. 14 is a side view of first tubular member 11 at bent portion 11e in a movable sheath 400.

### DESCRIPTION OF EMBODIMENTS

Details of the embodiment will be described with reference to the drawings. In the following drawings, the same or corresponding parts are denoted by the same reference numerals, and redundant description will not be repeated.

### First Embodiment

Hereinafter, a configuration of a movable sheath (hereinafter referred to as a “movable sheath 100”) according to a first embodiment will be described.

<Schematic Configuration of Movable Sheath 100>

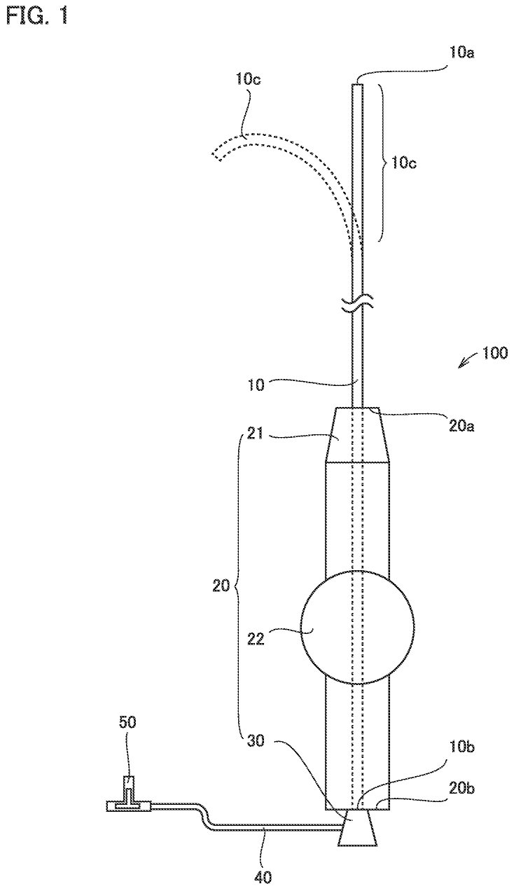

FIG. 1 is a plan view of movable sheath 100. As illustrated in FIG. 1, movable sheath 100 includes a movable shaft 10, a hand operation portion 20, a hemostasis valve 30, a tube 40, and a three-way stopcock 50. Movable shaft 10 has a distal end 10a and a proximal end 10b in a longitudinal direction of the movable shaft. Hand operation portion 20 is attached to proximal end 10b.

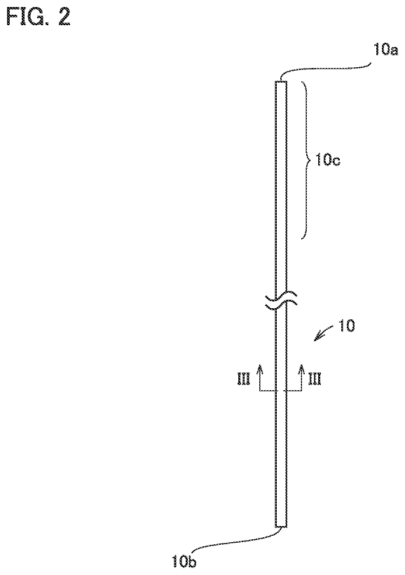

FIG. 2 is a plan view of movable shaft 10. Movable shaft 10 has a bent portion 10c. Bent portion 10c extends along the longitudinal direction of movable shaft 10. Movable shaft 10 bends at bent portion 10c by operating hand operation portion 20. In FIG. 1, movable shaft 10 bent by an operation of hand operation portion 20 is indicated by a dotted line.

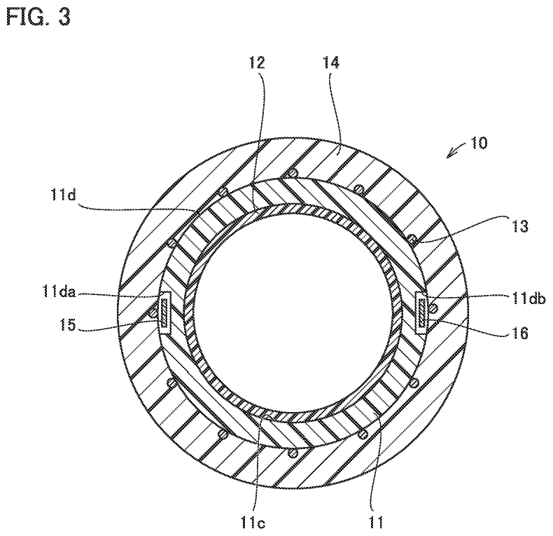

FIG. 3 is a cross-sectional view taken along line III-III in FIG. 2. As illustrated in FIG. 3, movable shaft 10 includes a first tubular member 11, a second tubular member 12, a braid 13, an outer sheath 14, a pull wire 15, and a pull wire 16.

First tubular member 11 is a tubular member. First tubular member 11 is preferably made of a polyether ether ketone resin (PEEK resin). First tubular member 11 may be made of liquid crystal polymer, nylon, polycarbonate, polyimide, silicone, polyvinyl chloride (PVC), polyethylene, or the like. A material constituting first tubular member 11 is not limited thereto.

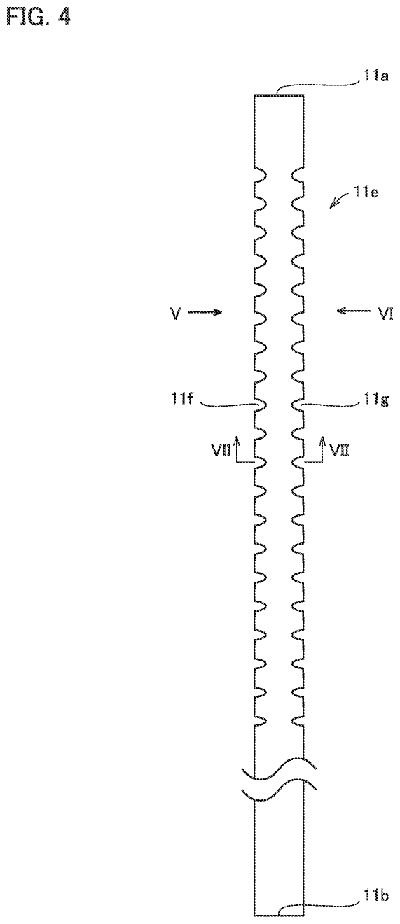

First tubular member 11 has a distal end 11a and a proximal end 11b in the longitudinal direction thereof (see FIG. 4). Distal end 11a is located on a side of distal end 10a side, and proximal end 11b is located on a side of proximal end 10b.

First tubular member 11 has an inner peripheral surface 11c and an outer peripheral surface 11d. On outer peripheral surface 11d, a first groove 11da and a second groove 11db are provided. First groove 11da and second groove 11db extend along the longitudinal direction of first tubular member 11. In first groove 11da and second groove 11db, outer peripheral surface 11d is recessed toward inner peripheral surface 11c. First groove 11da is located on a side opposite to second groove 11db across a central axis of first tubular member 11. Other configurations of first tubular member 11 will be described later.

Second tubular member 12 is inserted into first tubular member 11. Second tubular member 12 is a tubular member. When movable shaft 10 is applied to a movable sheath, an interior of second tubular member 12 is hollow. A guide wire, a catheter (for example, a catheter used for cardiac ablation treatment), and the like are inserted into second tubular member 12. The thickness of second tubular member 12 may be smaller than the thickness of first tubular member 11 (the thickness of first tubular member 11 may be thicker than the thickness of second tubular member 12). Second tubular member 12 is a tubular member.

Second tubular member 12 is made of, for example, a fluorine-based thermoplastic resin such as a polytetrafluoroethylene resin (PTFE resin), a PEEK resin, a polyvinylidene fluoride resin (PVDF resin), and a perfluoroalkoxy fluorine resin (PFA resin). However, the material constituting second tubular member 12 is not limited thereto, and may be made of a resin that satisfies lubricity in operation by a guide wire, a catheter, or the like inserted inside.

Braid 13 has a structure in which a metal wire is woven into a net shape. Braid 13 is disposed so as to cover outer peripheral surface 11d. A wire constituting braid 13 is made of, for example, stainless steel. Outer sheath 14 is disposed so as to cover outer peripheral surface 11d and braid 13. Outer sheath 14 is made of, for example, a fluororesin. A material constituting outer sheath 14 is not limited thereto, and any biocompatible material can be applied.

Pull wire 15 is disposed in groove 11da so as to be slidable along the longitudinal direction of first tubular member 11. Pull wire 15 is disposed along the longitudinal direction of first tubular member 11 while facing a row of first through holes 11f described later. Pull wire 16 is disposed in the groove 11db so as to be slidable along the longitudinal direction of first tubular member 11. Pull wire 16 is disposed along the longitudinal direction of first tubular member 11 while facing a row of second through holes 11g to be described later. Pull wire 15 and pull wire 16 are made of, for example, stainless steel. One end of pull wire 15 and one end of pull wire 16 are fixed to distal end 10a.

<Detailed Configuration of First Tubular Member 11>

FIG. 4 is an enlarged plan view of bent portion 11e of first tubular member 11. As illustrated in FIG. 4, first tubular member 11 has bent portion 11e. Bent portion 11e extends along the longitudinal direction of first tubular member 11. Bent portion 11e is at a position corresponding to bent portion 10c in a state of being disposed inside movable shaft 10. First tubular member 11 is allowed to be bent along the longitudinal direction of first tubular member 11 by the operation of pull wire 15 and pull wire 16 (hand operation portion 20) at bent portion 11e.

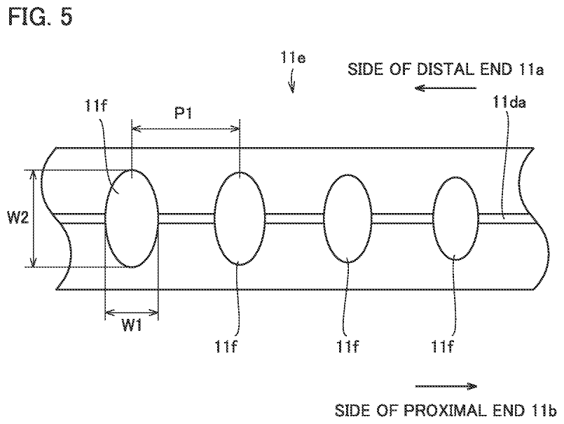

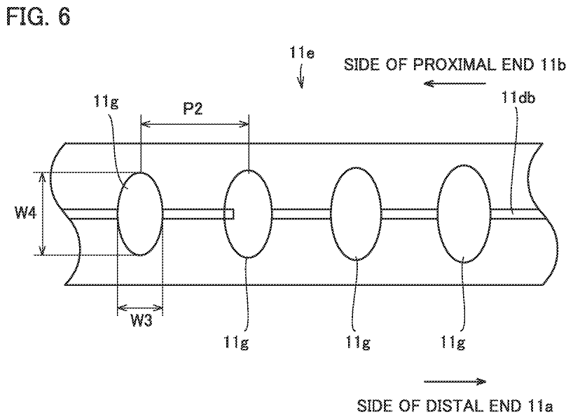

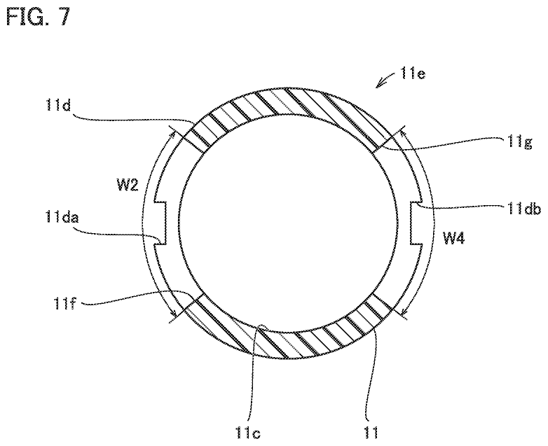

FIG. 5 is a side view of first tubular member 11 as viewed from a direction V in FIG. 4. FIG. 6 is a side view of first tubular member 11 as viewed from a direction VI in FIG. 4. FIG. 7 is a cross-sectional view taken along line VII-VI in FIG. 4. As illustrated in FIGS. 4, 5, 6, and 7, bent portion 11e is provided with a plurality of first through holes 11f and a plurality of second through holes 11g. First through holes 11f and second through holes 11g are opposite to each other with respect to a central axis of first tubular member 11.

First through holes 11f penetrate first tubular member 11 along the direction from outer peripheral surface 11d toward inner peripheral surface 11c. First through holes 11f are disposed in a row along the longitudinal direction of first tubular member 11.

The shape of first through holes 11f is, for example, an elliptical shape. A major axis of the elliptical shape is along a circumferential direction of first tubular member 11, and a minor axis of the elliptical shape is along the longitudinal direction of first tubular member 11. The width of first through holes 11f in the longitudinal direction of first tubular member 11 is defined as a width W1. The width of first through holes 11f in the circumferential direction of first tubular member 11 is defined as a width W2. A pitch between two first through holes 11f adjacent to each other in the longitudinal direction of first tubular member 11 is defined as a pitch P1. Width W1 and/or width W2 increases from the side of proximal end 11b toward the side of distal end 11a. Pitch P1 is constant, for example.

The shape of second through holes 11g is, for example, an elliptical shape. A major axis of the elliptical shape is along a circumferential direction of first tubular member 11, and a minor axis of the elliptical shape is along the longitudinal direction of first tubular member 11. The width of second through holes 11g in the longitudinal direction of first tubular member 11 is defined as a width W3. The width of second through holes 11g in the circumferential direction of first tubular member 11 is defined as a width W4. A pitch between two second through holes 11g adjacent to each other in the longitudinal direction of first tubular member 11 is defined as a pitch P2. Width W3 and/or width W4 increases from the side of proximal end 11b toward the side of distal end 11a. Pitch P2 is constant, for example.

Although not illustrated, first through holes 11f and second through holes 11g are closed by second tubular member 12 from a side of inner peripheral surface 11c.

In the above example, width W1 and/or width W2 (width W3 and/or width W4) is increased from the side of proximal end 11b toward the side of distal end 11a, and pitch P1 (pitch P2) is constant. However, pitch P1 (pitch P2) may be decreased from the side of proximal end 11b toward the side of distal end 11a, and width W1 and/or width W2 (width W3 and/or width W4) may be constant. In addition, width W1, width W2, width W3, and width W4 may be increased from the side of proximal end 11b toward the side of distal end 11a.

Since first through holes 11f and second through holes 11g are provided in bent portion 11e, flexibility is imparted to bent portion 11e. Since width W1 and/or width W3 increases from the side of proximal end 11b toward the side of distal end 11a, the flexibility of bent portion 11e increases from the side of proximal end 11b toward the side of distal end 11a.

<Modification of First Tubular Member 11>

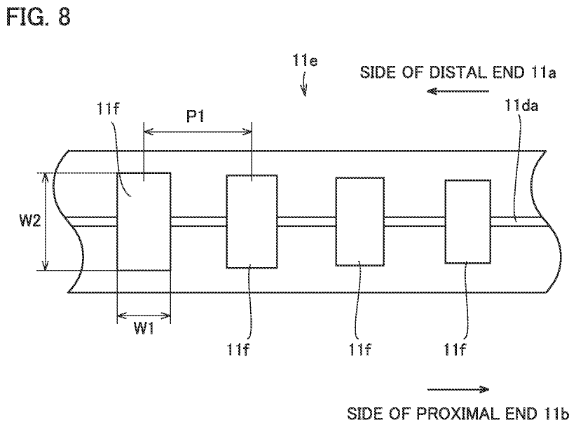

FIG. 8 is a side view of first tubular member 11 according to a first modification. As illustrated in FIG. 8, first through holes 11f and second through holes 11g (not illustrated) may have, for example, a rectangular shape. A long side of the rectangular shape is along the circumferential direction of first tubular member 11, and a short side of the rectangular shape is along the longitudinal direction of first tubular member 11.

A case where corners of first through holes 11f and second through holes 11g (not illustrated) are rounded is also included in the “rectangular shape”. In first tubular member 11 according to the first modification, since first through holes 11f and second through holes 11g (not illustrated) are provided in bent portion 11e, flexibility is imparted to bent portion 11e. Although not illustrated, first through holes 11f and second through holes 11g (not illustrated) are closed by second tubular member 12.

In first tubular member 11 according to the first modification, width W1 and/or width W2 (width W3 (not illustrated) and/or W4 (not illustrated)) increases from the side of proximal end 11b toward the side of distal end 11a, and pitch P1 (pitch P2 (not illustrated)) is constant, for example. As a result, the flexibility of bent portion 11e increases from the side of proximal end 11b toward the side of distal end 11a.

In the above example, width W1 and/or width W2 (width W3 and/or width W4) is increased from the side of proximal end 11b toward the side of distal end 11a, and pitch P1 (pitch P2) is constant. However, pitch P1 (pitch P2) may be decreased from the side of proximal end 11b toward the side of distal end 11a, and width W1 and/or width W2 (width W3 and/or width W4) may be constant. In addition, width W1, width W2, width W3, and width W4 may be increased from the side of proximal end 11b toward the side of distal end 11a.

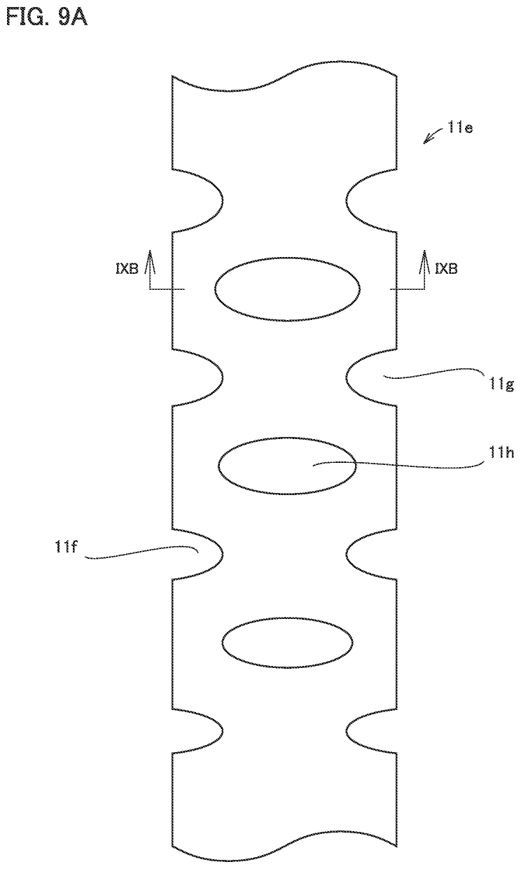

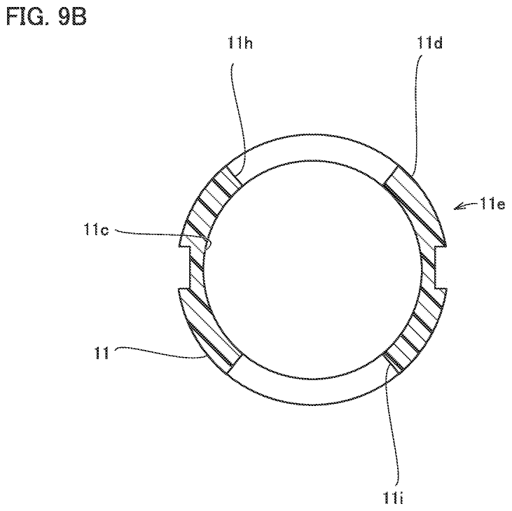

FIG. 9A is an enlarged plan view of first tubular member 11 according to a second modification. FIG. 9B is a cross-sectional view taken along line IXB-IXB in FIG. 9A. First tubular member 11 may be provided with a plurality of third through holes 11h and a plurality of fourth through holes 11i.

Third through holes 11h and fourth through holes 11i penetrate first tubular member 11 along the direction from the outer peripheral surface 11d toward inner peripheral surface 11c. Third through holes 11h are provided in a row along the longitudinal direction of first tubular member 11, and fourth through holes 11i are provided in a row along the longitudinal direction of first tubular member 11. Third through holes 11h are each disposed between two first through holes 11f (second through holes 11g) adjacent to each other in the longitudinal direction of first tubular member 11, and fourth through holes 11i are each disposed between two first through holes 11f (second through holes 11g) adjacent to each other in the longitudinal direction of first tubular member 11.

Third through holes 11h and fourth through holes 11i are arranged to face each other in a direction orthogonal to the direction from first through holes 11f toward second through holes 11g. The widths of third through holes 11h and fourth through holes 11i in the longitudinal direction of first tubular member 11 increase from the side of proximal end 11b toward the side of distal end 11a. The pitch of two third through holes 11h adjacent to each other in the longitudinal direction of first tubular member 11 and the pitch of two fourth through holes 11i adjacent to each other in the longitudinal direction of first tubular member 11 are, for example, constant. Although not illustrated, first through holes 11f, second through holes 11g, third through holes 11h, and fourth through holes 11i are closed by second tubular member 12.

In first tubular member 11 according to the second modification, the pitch of two third through holes 11h adjacent in the longitudinal direction of first tubular member 11 and/or the pitch of two fourth through holes 11i adjacent in the longitudinal direction of first tubular member 11 may decrease from the side of proximal end 11b toward the side of distal end 11a.

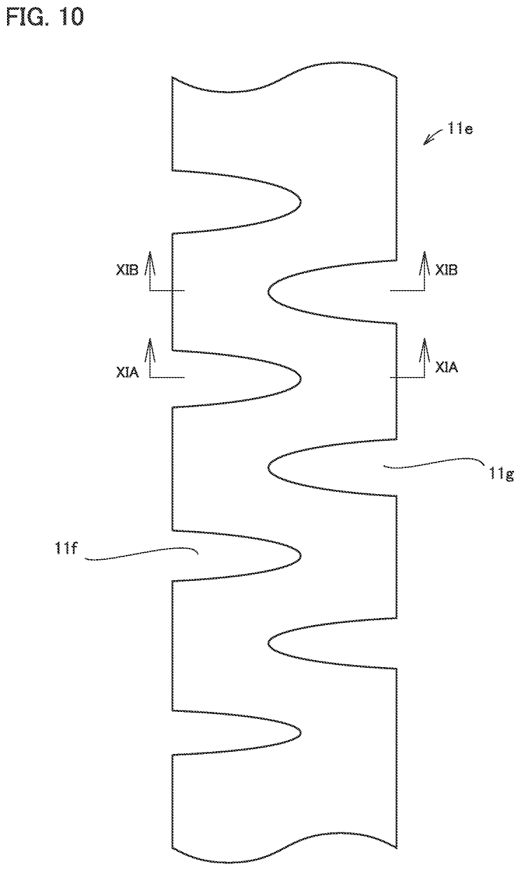

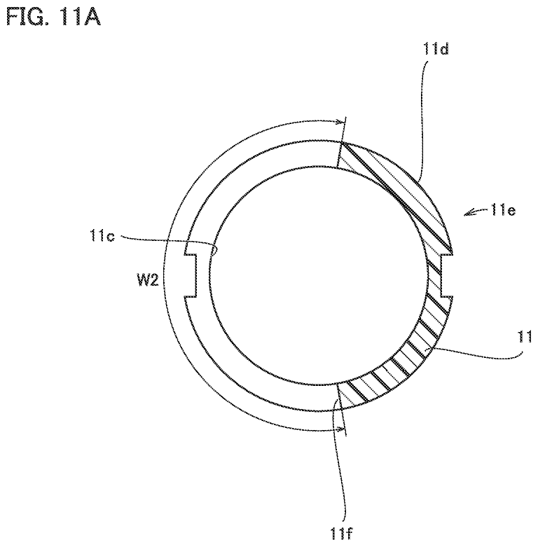

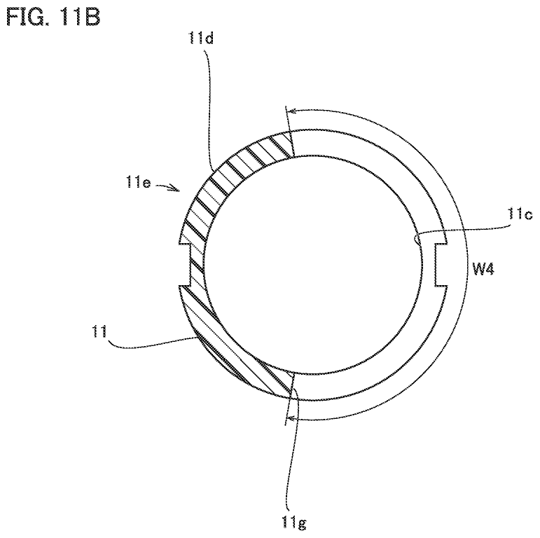

FIG. 10 is an enlarged plan view of first tubular member 11 according to a third modification. As illustrated in FIG. 10, second through holes 11g are each disposed between two first through holes 11f adjacent to each other in the longitudinal direction of first tubular member 11. The row of first through holes 11f and the row of second through holes 11g are opposite to each other with respect to the central axis of first tubular member 11. That is, first through holes 1 if and second through holes 11g are provided stepwise in the longitudinal direction of first tubular member 11. FIG. 11A is a cross-sectional view taken along line XIA-XIA in FIG. 10. FIG. 11B is a cross-sectional view taken along line XIB-XIB in FIG. 10. As illustrated in FIGS. 11A and 11B, width W2 and width W4 may be 0.5 times larger than the outer peripheral length of first tubular member 11. As a result, the flexibility of bent portion 11e can be further enhanced.

In first tubular member 11 according to the third modification, width W1 (not illustrated) and/or width W2 (width W3 (not illustrated) and/or W4) increases from the side of proximal end 11b toward the side of distal end 11a, and pitch P1 (not illustrated) and/or pitch P2 (not illustrated) are constant, for example. In first tubular member 11 according to the third modification, pitch P1 (not illustrated) and/or pitch P2 (not illustrated) may decrease from the side of proximal end 11b toward the side of distal end 11a. Although not illustrated, first through holes 1 if and second through holes 11g are closed by second tubular member 12 from the side of inner peripheral surface 11c.

<Detailed Configuration of Hand Operation Portion 20>

As illustrated in FIG. 1, hand operation portion 20 has a first end 20a and a second end 20b. Second end 20b is an end opposite to first end 20a. Movable shaft 10 passes through an interior of hand operation portion 20, and proximal end 10b reaches second end 20b of hand operation portion 20. Hand operation portion 20 includes a gripping portion 21 and a drive unit 22.

Gripping portion 21 is a portion for the user to grip movable sheath 100 (hand operation portion 20). Drive unit 22 has, for example, a disk shape. Drive unit 22 is rotatable about the central axis. Although not illustrated, the other end of pull wire 15 and the other end of pull wire 16 are fixed to an outer peripheral surface of drive unit 22. The position on the outer peripheral surface of drive unit 22 to which the other end of pull wire 15 is fixed and the position on the outer peripheral surface of drive unit 22 to which the other end of pull wire 16 is fixed are point-symmetric with respect to the central axis of drive unit 22.

Rotation of the drive unit 22 about the central axis causes pull wire 15 to be pulled in along the direction from distal end 10a toward proximal end 10b and pull wire 16 to be pushed out along the direction from proximal end 10b toward distal end 10a, so that movable shaft 10 is bent into a shape indicated by a dotted line in FIG. 1. When drive unit 22 is reversely rotated about the central axis, pull wire 15 is pushed out along the direction from proximal end 10b toward distal end 10a, and pull wire 16 is pulled in along the direction from distal end 10a toward proximal end 10b, so that movable shaft 10 returns to its original shape. When drive unit 22 is further reversely rotated about the central axis, movable shaft 10 bends to the side opposite to the shape indicated by the dotted line in FIG. 1.

Hand operation portion 20 further includes a hemostasis valve 30. An interior of hemostasis valve 30 is hollow. Hemostasis valve 30 is attached to second end 20b. As a result, the interior of hemostasis valve 30 communicates with an interior of movable shaft 10 (more specifically, the interior of second tubular member 12). Hemostasis valve 30 is provided with an insertion port. A guide wire, a catheter, or the like is inserted into movable shaft 10 from this insertion port.

<Detailed Configuration of Tube 40 and Three-Way Stopcock 50>

Tube 40 is connected at one end to hemostasis valve 30. An interior of tube 40 communicates with the interior of hemostasis valve 30. Three-way stopcock 50 is attached to the other end of tube 40. By attaching a syringe (not illustrated) to three-way stopcock 50, removal of air or blood from the interior of movable shaft 10 or supply of a medicinal solution to the interior of movable shaft 10 is performed.

The effect of movable sheath 100 will be described below.

The interval between the opening edges of first through holes 11f (second through holes 11g) facing each other in the longitudinal direction of first tubular member 11 narrows as bent portion 11e bends, and finally, the opening edges of first through holes 11f (second through holes 11g) facing each other in the longitudinal direction of first tubular member 11 come into contact with each other.

When the opening edges of first through holes 11f (second through holes 11g) facing each other in the longitudinal direction of first tubular member 11 come into contact with each other, bending deformation of bent portion 11e is restricted at a position where first through holes 11f (second through holes 11g) with which the opening edges come into contact (hereinafter, the bending angle of bent portion 11e in this state is referred to as an “upper limit bending angle”).

As described above, width W1 (width W3) increases from the side of proximal end 11b toward the side of distal end 11a. Therefore, the upper limit bending angle of bent portion 11e increases from the side of proximal end 11b toward the side of distal end h a.

In a case where the upper limit bending angle of bent portion 11e decreases from the side of proximal end 11b toward the side of distal end 11a, when pull wire 15 (pull wire 16) is pulled by hand operation portion 20, bent portion 11e is greatly bent on proximal end 11b side. Therefore, in this case, the curved shape of bent portion 11e is not stable.

On the other hand, when the bending angle of bent portion 11e increases as the upper limit bending angle of bent portion 11e comes closer toward the side of distal end 11a from the side of proximal end 11b, the bending of bent portion 11e does not change greatly on the side of proximal end 11b even if pull wire 15 (pull wire 16) is pulled, so that the curved shape of bent portion 11e is stabilized.

As described above, in movable sheath 100, since width W1 (flexibility of bent portion 11e) increases from the side of proximal end 11b toward the side of distal end 11a, controllability of the curved shape in bent portion 11e can be improved.

### Second Embodiment

Hereinafter, a configuration of a movable sheath (hereinafter referred to as a “movable sheath 200”) according to a second embodiment will be described. Here, differences from the configuration of movable sheath 100 will be mainly described, and redundant description will not be repeated.

Movable sheath 200 includes movable shaft 10, hand operation portion 20, hemostasis valve 30, tube 40, and three-way stopcock 50. In movable sheath 200, first through holes 11f and second through holes 11g are provided in bent portion 11e, and the flexibility of bent portion 11e increases from the side of proximal end 11b toward the side of distal end 11a. In these respects, the configuration of movable sheath 200 is common to the configuration of movable sheath 100.

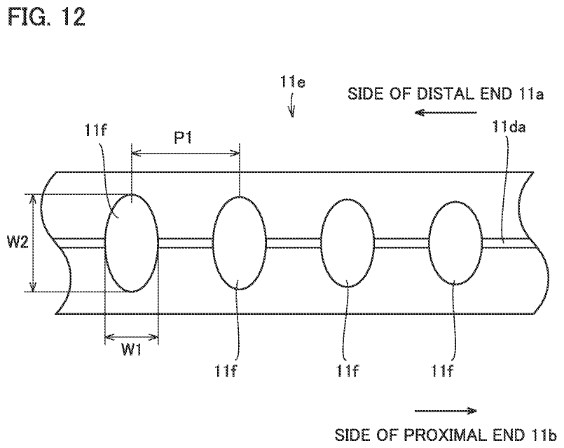

However, the configuration of movable sheath 200 is different from the configuration of movable sheath 100 in the arrangement and the shape of first through holes 11f and second through holes 11g (not illustrated). FIG. 12 is a side view of first tubular member 11 at bent portion 11e in movable sheath 200. As illustrated in FIG. 12, width W1 and pitch P1 are constant. Although not illustrated, width W3 (not illustrated) and pitch P2 (not illustrated) are also constant. Width W2 and/or width W4 (not illustrated) increases from the side of proximal end 11b toward the side of distal end 11a.

The effect of movable sheath 200 will be described below. Here, differences from the effect of movable sheath 100 will be mainly described, and redundant description will not be repeated.

In movable sheath 200, width W2 (width W4) increases from the side of proximal end 11b toward the side of distal end 11a, and the flexibility of bent portion 11e increases from the side of proximal end 11b toward the side of distal end 11a.

Therefore, according to movable sheath 200, similarly to movable sheath 100, controllability of the curved shape in bent portion 11e can be improved.

### Third Embodiment

Hereinafter, a configuration of a movable sheath (hereinafter referred to as a “movable sheath 300”) according to a third embodiment will be described. Here, differences from the configuration of movable sheath 100 will be mainly described, and redundant description will not be repeated.

Movable sheath 300 includes movable shaft 10, hand operation portion 20, hemostasis valve 30, tube 40, and three-way stopcock 50. In movable sheath 300, first through holes 11f and second through holes 11g are provided in bent portion 11e, and the flexibility of bent portion 11e increases from the side of proximal end 11b toward the side of distal end 11a. In these respects, the configuration of movable sheath 300 is common to the configuration of movable sheath 100.

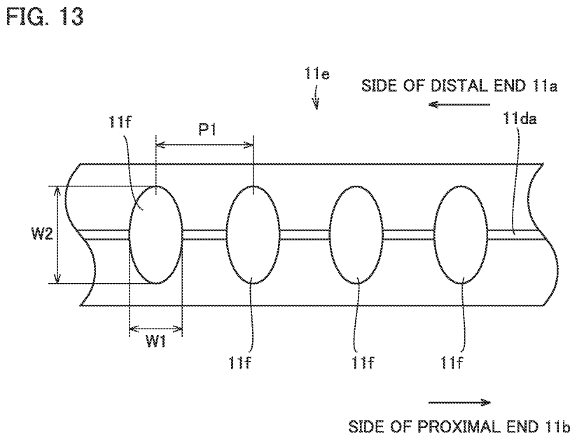

However, the configuration of movable sheath 300 is different from the configuration of movable sheath 100 in the arrangement and the shape of first through holes 11f and second through holes 11g. FIG. 13 is a side view of first tubular member 11 at bent portion 11e in movable sheath 300. As illustrated in FIG. 13, width W1, width W2, width W3 (not illustrated), and width W4 (not illustrated) are constant. Pitch P1 and/or pitch P2 (not illustrated) decreases from the side of proximal end 11b toward the side of distal end 11a.

The effect of movable sheath 300 will be described below. Here, differences from the effect of movable sheath 100 will be mainly described, and redundant description will not be repeated.

In movable sheath 300, pitch P1 (pitch P2) decreases from the side of proximal end 11b toward the side of distal end 11a, and the flexibility of bent portion 11e increases from the side of proximal end 11b toward the side of distal end 11a.

Therefore, according to movable sheath 300, similarly to movable sheath 100, controllability of the curved shape in bent portion 11e can be improved.

### Fourth Embodiment

Hereinafter, a configuration of a movable sheath (hereinafter referred to as a “movable sheath 400”) according to a fourth embodiment will be described. Here, differences from the configuration of movable sheath 100 will be mainly described, and redundant description will not be repeated.

Movable sheath 400 includes movable shaft 10, hand operation portion 20, hemostasis valve 30, tube 40, and three-way stopcock 50. In movable sheath 400, first through holes 11f and second through holes 11g are provided in bent portion 11e, and the flexibility of bent portion 11e increases from the side of proximal end 11b toward the side of distal end 11a. In these respects, the configuration of movable sheath 300 is common to the configuration of movable sheath 100.

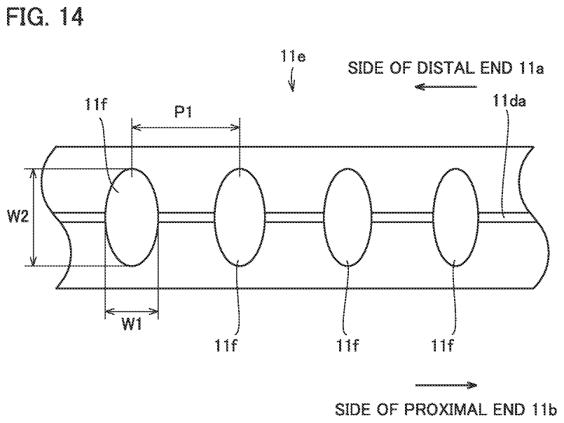

However, the configuration of movable sheath 400 is different from the configuration of movable sheath 100 in the arrangement and the shape of first through holes 1 if and second through holes 11g. FIG. 14 is a side view of first tubular member 11 at bent portion 11e in movable sheath 400. As illustrated in FIG. 14, width W2 and width W4 (not illustrated) and pitch P1 and pitch P2 (not illustrated) are constant.

Width W1 and/or width W3 (not illustrated) increases from the side of proximal end 11b toward the side of distal end 11a.

The effect of movable sheath 400 will be described below. Here, differences from the effect of movable sheath 100 will be mainly described, and redundant description will not be repeated.

In movable sheath 400, width W1 (and width W3 (not illustrated)) increases from the side of proximal end 11b toward the side of distal end 11a, and the flexibility of bent portion 11e increases from the side of proximal end 11b toward the side of distal end 11a. Therefore, according to movable sheath 400, similarly to movable sheath 100, controllability of the curved shape in bent portion 11e can be improved.

### Other Embodiments

In the above description, the movable sheath has been exemplified as the medical instrument having movable shaft 10, but the medical instrument having movable shaft 10 is not limited thereto. Other examples of the medical instrument having movable shaft 10 include a catheter and an endoscope.

Although the embodiments of the present invention have been described above, the above-described embodiments can be variously modified. In addition, the scope of the present invention is not limited to the above-described embodiments. The scope of the present invention is defined by the claims, and is intended to include meanings equivalent to the claims and all modifications within the scope.

### INDUSTRIAL APPLICABILITY

The present embodiments are particularly advantageously applied to a movable sheath into which a catheter for cardiac ablation treatment is inserted.

### REFERENCE SIGNS LIST

10: movable shaft; 20: hand operation portion; 20a: first end; 20b: second end; 30: hemostasis valve; 40: tube; 50: three-way stopcock; 10a: distal end; 10b: proximal end; 10c: bent portion; 11: first tubular member; 11a: distal end; 11b: proximal end; 11c: inner peripheral surface; 11d outer peripheral surface; 11da: first groove; 11db: second groove; 11e: bent portion; 11f: first through hole; 11g: second through hole; 11h: third through hole; 11i: fourth through hole; 12: second tubular member; 13: braid; 14: outer sheath; 15: pull wire; 16: pull wire; 21: gripping portion; 22: drive unit; P1, P2: pitch; W1, W2, W3, W4: width; 100, 200, 300, 400: movable sheath.

1. A movable shaft comprising a tubular member, wherein
the tubular member has a proximal end and a distal end in a longitudinal direction of the tubular member,
the tubular member has a bent portion extending along the longitudinal direction and allowed to be bent,
flexibility of the bent portion increases from a side of the proximal end to a side of the distal end,
the tubular member has an inner peripheral surface and an outer peripheral surface,
the bent portion includes a plurality of through holes disposed in a row at intervals along the longitudinal direction, the through holes penetrating the tubular member from the outer peripheral surface toward the inner peripheral surface, and
a pitch between two through holes adjacent to each other in the longitudinal direction decreases from the side of the proximal end to the side of the distal end.

2. (canceled)

3. A movable shaft comprising a tubular member, wherein
the tubular member has a proximal end and a distal end in a longitudinal direction of the tubular member,
the tubular member has a bent portion extending along the longitudinal direction and allowed to be bent,
flexibility of the bent portion increases from a side of the proximal end to a side of the distal end,
the tubular member has an inner peripheral surface and an outer peripheral surface,
the bent portion includes a plurality of through holes disposed in a row at intervals along the longitudinal direction, the through holes penetrating the tubular member from the outer peripheral surface toward the inner peripheral surface, and
at least one of a width of the through hole in the longitudinal direction and a width of the through hole in a circumferential direction of the tubular member increases from the side of the proximal end to the side of the distal end.

4. The movable shaft according to claim 1, further comprising:
a pull wire disposed along the longitudinal direction so as to face the through hole.

5. A medical instrument comprising:
the movable shaft according to claim 4, wherein
the bent portion is bent by pulling the pull wire.

6. The movable shaft according to claim 3, further comprising:
a pull wire disposed along the longitudinal direction so as to face the through hole.

7. A medical instrument comprising:
the movable shaft according to claim 6, wherein
the bent portion is bent by pulling the pull wire.
